(19) United States
(12) Patent Application Publication
(10) Pub. No.: US 2009/0062675 A1
(43) Pub. Date: Mar. 5, 2009

(54) DETECTION OF THE BEGINNING OF AN APNEA

(75) Inventors: Christian Weigand, Buckenhof (DE); Matthias Struck, Erlangen (DE)

Correspondence Address: SCHOPPE, ZIMMERMANN , STOCKELER & ZINKLER;C/O KEATING & BENNETT, LLP, 1800 Alexander Bell Drive, SUITE 200, Reston, VA, 20191, US

(73) Assignee: Fraunhofer-Gesellschaft zur Foerderung der angewandten Forschung e.V., Munich (DE)

(21) Appl. No.: 12/296,699

(22) PCT Filed: Mar. 28, 2007
(86) PCT No.: PCT/EP2007/002760

§ 371 Date: Nov. 14, 2008

(30) Foreign Application Priority Data
Apr. 12, 2006 (DE) 10 2006 017 278.7

(51) Int. Cl. A61B 5/08; A61B 7/00

(52) U.S. Cl. 600/529; 600/586

(57) ABSTRACT

The beginning of an apnea can be recognized reliably if a series of sample values describing the breathing noise of a patient are processed in block-wise manner, and if a fingerprint with a predetermined number of fingerprint coefficients describing a waveform of the sample values within a block is determined for a number of sample values within the block. Since the number of fingerprint coefficients is smaller than the number of sample values within the block, comparison of the fingerprint coefficients with reference fingerprint coefficients characteristic for the waveform at the beginning of an apnea can be performed efficiently and reliably, in order to detect the beginning of the apnea.

### BACKGROUND OF THE INVENTION

The present invention concerns the detection of sleep disorders, and particularly how the beginning of an apnea can be detected by means of digital signal processing.

Sleep disorders are a phenomenon occurring more and more frequently, heavily restricting the quality of life and capability of the peopled affected. Special types of occurring sleep disorders may further have a lasting detrimental effect on the patient's health.

Two sleep disorders occurring especially frequently are apneas and hypopneas. In case of an apnea, complete respiratory short-term arrests occur, the frequency of which may vary within wide boundaries, with values of more than 35 of such sleep disorders per night not being rare. The occurrence of at least 10 respiratory arrests, each lasting for at least 10 seconds, within one hour of sleep is regarded as the general definition for the disease of apnea. Apnea may have several causes, with the most frequent one being occlusion of the upper respiratory tracts occurring during sleep (obstructive sleep apnea). The occlusion normally is caused by relaxation of the soft palate (velum), which also is responsible for snoring, among other things. If the velum relaxes, it may lead to the fact that it completely closes the respiratory tracts, so that the supply of oxygen to the lungs, and hence also to the brain, is interrupted. Due to the above connection, apnea often also is observed in people prone to heavy snoring. Induced by the falling oxygen content of the blood, the heart rate decreases and the blood pressure drops. This decrease in vital parameters triggers an alarm signal or counter measure in the brain after a certain time, so that the people concerned experience a so-called arousal at the end of an apnea, for example triggered by increased adrenalin production. In case of an arousal, the patient concerned typically is startled with a loud snoring noise, whereupon breathing starts again. Heartbeat as well as oxygen content may normalize. As described above, since this process repeats several times per night, it becomes obvious that sleep apnea may cause a series of negative side effects, such as increased fatigue during the daytime, reduced mental and physical capability, lack of concentration, headache, depressions, and the like.

Apart from obstructive apnea, so-called central sleep apnea often also is observed, wherein no occlusion of the respiratory tracts takes place, but which rather is due to a cessation of the breathing impulses on the part of the brain. Here, the observable course of the apnea until the arousal substantially is the same as of the obstructive apnea.

A disease closely related to apnea is hypopnea, for which there is no unique classification. With hypopnea, the breathing volume is reduced heavily for various reasons during the sleep for the duration of the hypopnea, so that the hypopnea also leads to a reduction of the oxygen content in the blood as well as of the heart rate. Due to the same symptoms, the health damage that may be caused by hypopneas also is similarly severe, as explained above in the case of apneas. In contrast to apnea, however, it usually is not possible to observe the arousal, i.e. the vigorous short-term wakeup process, in hypopnea. Just like with apnea, however, patients who snore are affected by hypopnea in a clearly disproportionate way.

On the basis of FIGS. 5A and 5B, a typical waveform, as it occurs during the occurrence of an apnea and/or hypopnea, will be illustrated briefly in the following. FIG. 5A here describes an apnea event, and FIG. 5B a hypopnea event, wherein, in both illustrations, time is plotted on the X axis and the amplitude course of the breathing noise of a sleeping patient as recorded by means of a microphone on the Y axis.

FIG. 5A shows a normal sleeping rhythm in a first area 2, in which a slight snoring noise is detected at almost regular intervals. FIG. 5A further shows the apnea area 4, in which the respiratory arrest occurs, and within which no signal amplitude is recorded as a result thereof. In FIG. 5A, an arousal area 6, which is characterized in that the patient resumes breathing with loud snoring at the end of the apnea, as already described above, can be seen immediately after the apnea area 4, which is why higher amplitudes are recorded in the arousal area 6 than in the first area 2, in which the patient is still sleeping normally. Immediately before the apnea area 4, an indicator area 8 further is illustrated in FIG. 5A, preceding the apnea area 4, and within which the recorded amplitudes or recorded waveform clearly differ from the signals in the first area 2, in which the patient is in a normal sleeping phase. The indicator area is typical of the occurrence of an apnea event, which means that such a waveform typically is observed prior to the beginning of the respiratory arrest in the apnea area 4 for all patients. The acoustic impression approximately is that of short violent snoring, which may often be combined with a slight groaning noise. One possibility of detecting an apnea therefore consists in e.g. detecting such a waveform in the recorded snoring noise.

FIG. 5B shows the occurrence of hypopnea, wherein, in FIG. 5B, at first a sleeping area 10 may be identified, in which the patient is in a normal sleeping condition, and in which snoring and/or breathing noises of significant amplitude are recorded at almost equidistant intervals. In the hypopnea area 12, in which the flow of breathing is reduced strongly, as already described above, only an extraordinarily low breathing noise is recorded during a time interval of more than 30 seconds. Here, it is to be noted that no typical waveform, like the indicator area 8 of the apnea, precedes the hypopnea. This has been confirmed by observing a multiplicity of hypopnea events in different patients.

Conventionally, a series of methods are described, which are applied to detect the beginning of an apnea in automated manner. The American patent application US 2004/0225226A1 and the U.S. Pat. No. 6,935,335B1 describe a method in which one or more microphones are employed, which pass the signals recorded thereby on to digital signal processing capable of detecting the beginning of an apnea event. The signal processing to this end performs a Fourier transform in the frequency domain and determines, through analysis of a great number of Fourier coefficients, if a waveform from which the beginning of an apnea event can be inferred is present. This method has the great disadvantage that a very large number of Fourier coefficients is generated by the Fourier analysis as a representation of the recorded signal. Thereby, real-time processing is made significantly more difficult, because a simple criterion indicating the occurrence of an apnea cannot be found if the multiplicity of the Fourier coefficients has to be employed for determining such a criterion.

European patent EP 0504945B1 describes how apnea events can be detected when both breathing and heart-rate tones are recorded. Substantially, a threshold value comparison is performed here for evaluating the recorded tones. This means that an apnea is assumed if one of the signals exceeds or falls short of a certain predetermined limit. Here, the threshold value comparison may additionally be performed in a frequency-effective manner by breaking down the recorded signal into fixed frequency ranges, with each frequency range possibly having its own threshold value. The method described here has the disadvantage that a threshold value comparison can only employ a single criterion, namely the energy value underlying the threshold value comparison, to detect the occurrence of an apnea. Using this single integral information here usually does not allow for recognizing the characteristic waveform, which does indeed not only distinguishes itself by its integrated intensity, with sufficiently high reliability prior to the beginning of an apnea.

U.S. Pat. No. 5,123,425 describes a collar suited to recognize and treat apnea events, with a microphone being used as sensor. Recognizing an apnea event also is done by simple threshold value excess here, so that the same disadvantages as already described above have to be accepted.

German patent specification DE 69632015T2 describes a sleep apnea treatment apparatus by means of which the ventilation pressure of a breathing mask can be adapted variably to the sleeping condition of a patient. Here, for detection of the sleeping condition, a sensor such as a microphone is used, recording a breathing signal within the frequency range from 20 Hz to 20,000 KHz and dynamically changing the breathing pressure on the basis of this signal for avoiding apnea events.

European patent application 0371424A1 describes a monitoring apparatus for the diagnosis of apnea, wherein both the heart rate and breathing noises are recorded, and wherein the onset of an apnea event is inferred from simple threshold value comparison both of the heart rate and the breathing loudness.

The methods described, which are based on a simple threshold value comparison, have the great disadvantage in the apnea detection that only an integral value is used as a criterion as to whether apnea has started or not. Hence, reliable detection usually is not possible, because the characteristic waveform has to be taken into account for this, which is not possible due to the integral property in the threshold value comparison.

Regarding the detection of hypopneas, the threshold value method has the great disadvantage that a fixed threshold value cannot reliably discover hypopnea, since it is characterized in that, during the occurrence of the hypopneas, there still exists a breathing noise the loudness of which may vary as compared with the normal breathing loudness, and which furthermore strongly depends on the patient.

The detection of a beginning of an apnea by means of Fourier analysis has the great disadvantage that, by the Fourier analysis, there is generated a multiplicity of Fourier coefficients describing the frequency spectrum of the recorded noise. A simple test or characterization of these Fourier coefficients, and thus capable of being performed within reasonable computation time, hardly is possible in real time due to the great number thereof. The complexity of the characterization prevents an apnea to be predicted already prior to the occurrence of the respiratory arrest thereof.

### SUMMARY

According to an embodiment, an apparatus for detecting the beginning of an apnea, using a series of sample values determined at predetermined time instants, which describe a breathing noise of a patient, may have: an analyzer for analyzing the series of sample values in block-wise manner to determine, for a number of sample values within a block corresponding to a time interval of the breathing noise, a fingerprint with a predetermined number of LPC coefficients describing a waveform of the sample values within the block, with the predetermined number of LPC coefficients being smaller than the number of sample values within the block; an evaluator formed to recognize the beginning of an apnea by comparison of a vector of the LPC coefficients with a vector of predetermined reference LPC coefficients characteristic of a waveform at the beginning of an apnea, wherein the beginning of an apnea is recognized if the vector of LPC coefficients lies within a tolerance range around the vector of reference LPC coefficients; and an alarm for performing an alarm action when the evaluator has recognized the beginning of an apnea.

According to another embodiment, a method of detecting the beginning of an apnea, using a series of sample values determined at predetermined time instants, which describe a breathing noise of a patient, may have the steps of: analyzing the series of sample values in block-wise manner to determine, for a number of sample values within a block corresponding to a time interval of the breathing noise, a fingerprint with a predetermined number of LPC coefficients describing a waveform of the sample values within the block, with the predetermined number of LPC coefficients being smaller than the number of sample values within the block; comparing a vector of the number of LPC coefficients with a vector of predetermined reference LPC coefficients characteristic for a waveform at the beginning of an apnea, in order to recognize the beginning of the apnea, wherein the beginning of an apnea is recognized if the vector of LPC coefficients lies within a tolerance range around the vector of reference LPC coefficients; and performing an alarm action at the beginning of an apnea.

According to another embodiment, a computer program may have a program code for performing, when the program is executed on a computer, a method of detecting the beginning of an apnea, using a series of sample values determined at predetermined time instants, which describe a breathing noise of a patient, wherein the method may have the steps of: analyzing the series of sample values in block-wise manner to determine, for a number of sample values within a block corresponding to a time interval of the breathing noise, a fingerprint with a predetermined number of LPC coefficients describing a waveform of the sample values within the block, with the predetermined number of LPC coefficients being smaller than the number of sample values within the block; comparing a vector of the number of LPC coefficients with a vector of predetermined reference LPC coefficients characteristic for a waveform at the beginning of an apnea, in order to recognize the beginning of the apnea, wherein the beginning of an apnea is recognized if the vector of LPC coefficients lies within a tolerance range around the vector of reference LPC coefficients; and performing an alarm action at the beginning of an apnea.

The present invention is based on the finding that the beginning of an apnea can be recognized reliably if a series of a sample values describing the breathing noise of a patient are processed in block-wise manner, and if a fingerprint with predetermined number of fingerprint coefficients, which describes a waveform of the sample values within the block, is determined for a number of sample values within a block. Since the number of fingerprint coefficients is smaller than the number of sample values within the block, comparison of the fingerprint coefficients with reference fingerprint coefficients characteristic for the waveform at the beginning of an apnea can be performed efficiently and reliably so as to detect the beginning of an apnea.

In one embodiment of the present invention, the medical finding that a characteristic signal within the breathing noise can be recognized in the by far greatest number of patients prior to the beginning of an apnea event is used to extract fingerprint coefficients describing the waveform at the beginning of the apnea with algorithms adapted from the field of automated speech processing. In an embodiment of the present invention, linear prediction is used for the extraction of the fingerprint coefficients. This method (LPC=linear predictive coding) here is particularly suited, because the mathematical method is motivated by the sound generation in the human pharyngeal space. Therefore, it is particularly suited to model and recognize all sounds generated by means of the human vocal organ. This also applies for snoring noises, which are not unlike the sounds prior to the beginning of an apnea event.

In the LPC method, the signal is processed in portions, in discrete time portions, that is. Here, LPC coefficients as fingerprint coefficients are extracted for each discrete time portion. The extraordinarily great advantage here lies in the fact that a very small number of LPC coefficients (8 or less LPC coefficients may already be sufficient, depending on the requirement) is generated from a great number of sample values (for example 4000), wherein characteristic waveforms occurring within the time window considered find their equivalent in the LPC coefficients.

The reduction in the number of parameters (fingerprint coefficients) describing the signal here is immediately accompanied by information loss. In contrast to conventional methods, which use an energy threshold value for the detection of an apnea, the method according to the invention, however, has the great advantage here that the information content is not reduced to only a single parameter. By applying the LPC coding, in particular, the reduction of the parameters may take place in a manner optimally suited for the modulation of the human vocal tract.

The decision as to whether an apnea event is impending or not is made on the basis of the fingerprint coefficients. This has the great advantage that the small number of fingerprint coefficients can be assessed reasonably and quickly with a criterion indicating the occurrence of an apnea.

In a further embodiment of the present invention, a Hidden-Markov model, also derived from the speech processing, is used for the extraction of the fingerprint coefficients. The Hidden-Markov model also is suited for applications in speech recognition and thus also is perfectly suited for the recognition of characteristic waveforms in noises generated by the human vocal tract. Above-indicated advantages thus also apply for the implementation by means of the Hidden-Markov model.

A simple criterion is utilized in a further embodiment of the present invention, wherein the occurrence of an apnea is assumed if the fingerprint coefficients have a Euclidian distance to a set of reference fingerprint coefficients lying below a predetermined and suitably chosen threshold value. The substantially occurring square subtraction of discrete numbers can be performed with very little computational effort, so that the decision can be made correspondingly quickly.

This is an advantage of the method according to the invention that is not to be underestimated, because it is the aim of the apnea detection to detect an apnea not only when it has already occurred, but to be able to detect it already at the beginning of the apnea with high significance, so that there may be the chance of still preventing the onset of the apnea.

In order to prevent the onset of the apnea, in a further embodiment of the present invention, an apparatus for detecting the beginning of an apnea is connected to alarm means capable of executing a plurality of alarm operations in the case of a detected beginning of an apnea. This may for example be alarming medical staff and/or stimulating the pharyngeal space of the patient, so as to completely or partially prevent the occurrence of the apnea, or controlling a device, such as a CPAP device.

### BRIEF DESCRIPTION OF THE DRAWINGS

Embodiments of the present invention will be detailed subsequently referring to the appended drawings, in which:

FIG. 1 shows an example of an apparatus for detecting the beginning of an apnea;

FIG. 2 shows an example of the block-wise processing of a series of sample values;

FIG. 3 is a flowchart for describing the method according to the invention;

FIG. 4 shows an example as to how reference fingerprint coefficients can be generated according to the invention;

FIG. 5A shows an example of the course of an apnea event; and

FIG. 5B shows an example of the course of a hypnoea event.

### DETAILED DESCRIPTION OF THE INVENTION

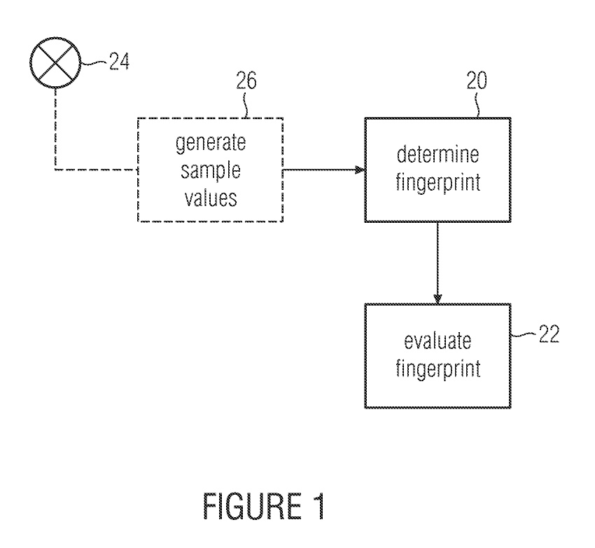

FIG. 1 shows an example of an apparatus according to the invention for detecting the beginning of an apnea, including an analyzer 20 as well as evaluation means 22. Moreover, FIG. 1 shows an optional microphone 24 connected to sampling means 26, which also is optional.

The analyzer 20 determines a predetermined number of fingerprint coefficients for a number of sample values corresponding to a time interval of the breathing noise, which may in principle be chosen freely, from the series of sample values. In an embodiment of the present invention, the length of this time interval, however, lies between 100 ms and 500 ms, because it has been realized that a typical time duration for an event preceding apnea is 200 ms.

In the following, the use of LPC coefficients as fingerprint coefficients is to be assumed exemplarily for illustrating the inventive concept. In the LPC, a (k+1)^{th} sample value is formed as a linear combination of the k sample values preceding the current sample value, wherein the LPC coefficients are those coefficients aᵢ for which the error of the linear prediction

$${f}_{k + 1} = {\sum }_{i = 1}^{k} {a}_{i} {f}_{i}$$

becomes minimum. In other words, the aᵢ are varied until the difference of the prediction value calculated according to this equation to the actual value fₖ₊ᵢ is minimum.

If the signal is processed in block-wise manner according to the invention, and a single signal block (time window) has n sample values, a total of n-k of the sample values can be described by linear prediction. In this case, the following linear system of equations is to be solved per time window:

$$( \begin{matrix}{f}_{k} & {f}_{k - 1} & … & {f}_{1} \\ {f}_{k + 1} & {f}_{k} & … & {f}_{2} \\ ⋮ & ⋮ & ⋱ & ⋮ \\ {f}_{n - 1} & {f}_{n - 2} & ⋯ & {f}_{n - k}\end{matrix} ) \cdot ( \begin{matrix}{a}_{1} \\ {a}_{2} \\ ⋮ \\ {a}_{k}\end{matrix} ) = ( \begin{matrix}{f}_{k + 1} \\ {f}_{k + 2} \\ ⋮ \\ {f}_{n}\end{matrix} )$$

This is possible with high efficiency with conventional methods, such as the singular value decomposition (SVD). A set of k LPC coefficients, which are characteristic for a mean waveform observed in the time window, thus is determined by the analyzer 20 according to the above formula per time window or block. By changing the width of the signal window, the method according to the invention may further be adapted to the specific noise patterns to be detected. In an embodiment of the present invention, the window width ranges from 100 to 500 ms, since it was realized that this is the typical time scale an event preceding apnea comprises.

The LPC coefficients (fingerprint coefficients) are communicated to the evaluating means 22 comparing same with a reference criterion, wherein, upon meeting the reference criterion, it is concluded that the momentary time window includes a signal indicating the beginning of an apnea.

As already described above, the number of the fingerprint coefficients can be varied freely, with the general provision that a higher number of coefficients can characterize a typical waveform more accurately. It has been realized, however, that LPC coefficients can describe a waveform characteristic for the beginning of an apnea so well that already a small number of coefficients (for example≦12) of LPC coefficients is sufficient to be able to reliably detect the beginning of an apnea.

The fingerprint coefficients determined by the analyzer 20 are communicated to the evaluating means 22, comparing same with reference fingerprint coefficients characteristic for a waveform at the beginning of an apnea. The great advantage here is that the number of the coefficients to be used for the comparison is substantially smaller than the number of the sample values underlying the coefficients, so that this comparison can be performed easily and in real time. Considering the fingerprint coefficients and/or the reference fingerprint coefficients each as a vector, a suitable criterion, for example, is the Euclidian distance between two vectors c and cⱼ, which is defined as follows:

d(cᵢ,cⱼ)=∥c−cⱼ∥²

Due to the small number of the fingerprint coefficients, this calculation can be performed easily and quickly, so that only very little computation latency time has to be put up with after the occurrence of the apnea event until the occurrence of the apnea is recognized.

Apart from the above-mentioned Euclidian distance, of course also other classificators can be used to decide, on the basis of the fingerprint coefficients, whether a waveform describing the beginning of an apnea is present or not.

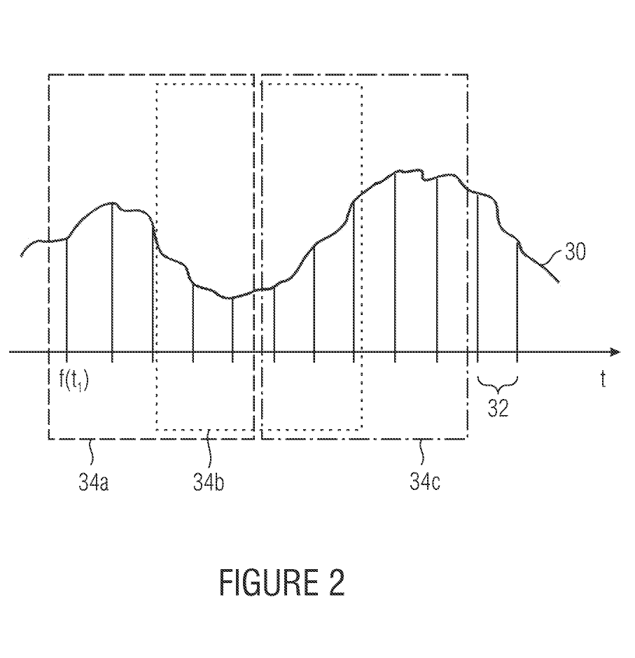

On the basis of FIG. 2 an example for the block and/or time-window-wise processing of sample values is shown. Time in arbitrary units is illustrated on the x-axis, and the amplitude of a signal 30 also in arbitrary units on the y-axis.

As can be seen on the basis of FIG. 2, the waveform is sampled at equidistant time intervals 32, i.e. the amplitude f(tᵢ) is determined and stored at the time instants tᵢ each. FIG. 2 exemplarily shows 3 time windows 34a, 34b and 34c, within which the respective amplitude values are processed in block-wise manner. In other words, this means that the amplitude values each located within a time window are used for determining the fingerprint coefficients. Here, the small number of the coefficients within a window only is chosen for simplicity reasons. For a reasonable application, the coefficients per time window typically are a lot more numerous. As already mentioned above, if time windows of several hundreds of ms length are chosen, which represent a reasonable time range for the signal to be sought, and if sample frequencies of 5 to 25 kHz are chosen, as it has proven to be extremely advantageous, several thousands of sample values are to be taken into account per time window.

As can be seen in FIG. 2, the time windows are arranged so that they overlap by half their width each. This may be necessary to really completely cover the area in which the event sought occurs with a window. If the stored event at the beginning of an apnea for example covered the boundaries of two non-overlapping windows (34a and 34c), safe detection by means of the reference fingerprint coefficients might no longer be guaranteed, since these were acquired by training or analysis of a plurality of events lying within a window.

It is not absolutely necessary, however, that the overlap exactly amounts to the half each, with arbitrary overlaps of the window areas being possible instead.

In a further embodiment of the present invention, effects at the edges of the time windows additionally are suppressed by providing all coefficients within a time window with statistical weights so that the coefficients located at the edges contribute less in the determination of the fingerprint coefficients. The manner in which these coefficients are chosen within the window is highly flexible here, with rectangular windows, Hamming windows and Hann windows being possible, for example.

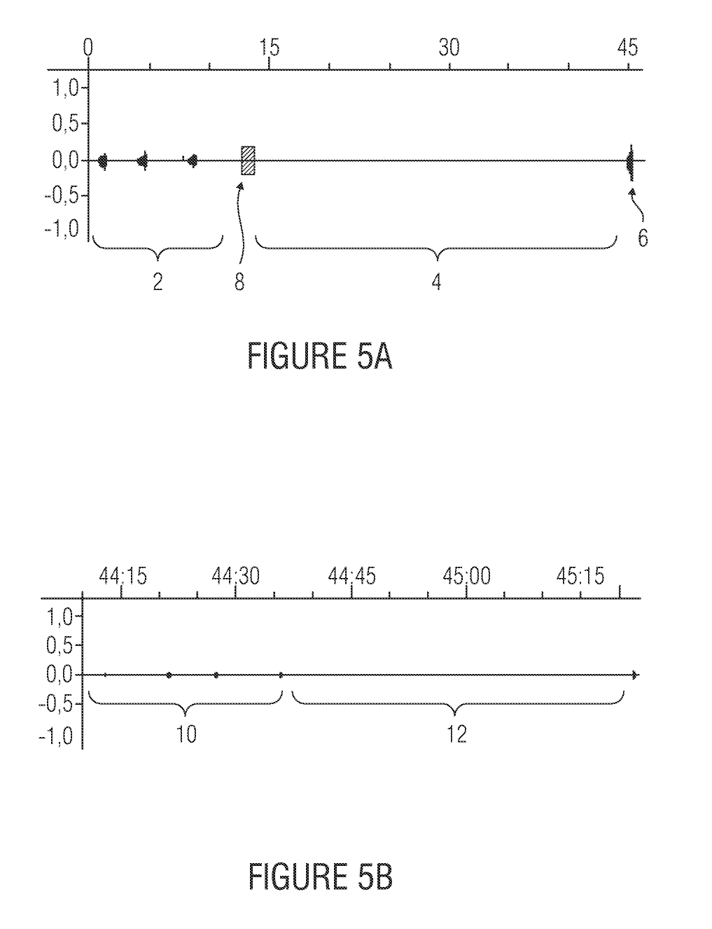

In an advantageous temporal overlap of the individual time windows, however, it is guaranteed that, given reasonable widths of the time windows, there is a time window each completely covering the waveform as it occurs in the area 8 marked in FIG. 5a.

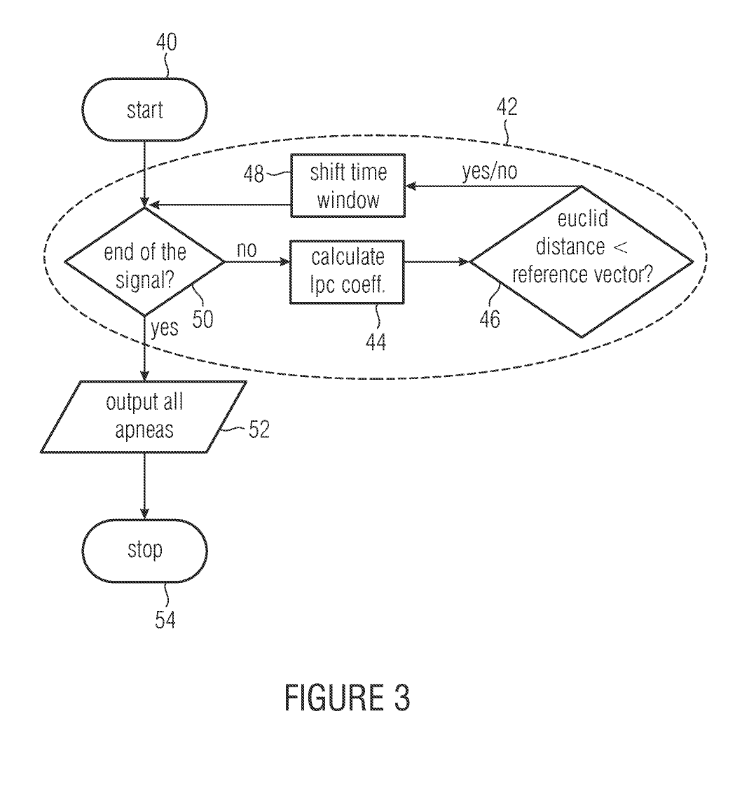

FIG. 3 shows a flowchart describing how the beginning of an apnea and/or several apneas can be detected using a series of sample values, according to the invention. At the beginning, the sample values are made available in the starting step 40. Then, an analysis loop 42 is commenced, in which at first a first time window at the beginning of the series of sample values is defined, from which the fingerprint coefficients are determined in an analysis step 44. In an evaluating step 46, it is checked whether the Euclidian distance between the fingerprint coefficients and the reference fingerprint coefficients is smaller than a predetermined value. If this is the case, a number of detected apneas is increased by one. In any case, the time window is shifted further by a predetermined number of sample values in an iteration step 48. In a checking step 50, it is checked whether the end of the time window now coincides with the end of the sample values, and/or goes beyond same. If this is the case, the analysis is completed, and the number of the detected apneas is output in an output step 52.

In a final step 54, the program execution then is stopped.

Altogether, the analysis loop 42 is passed through until all sample values made available have been taken into account in the calculation and/or detection of waveforms indicating a beginning of an apnea.

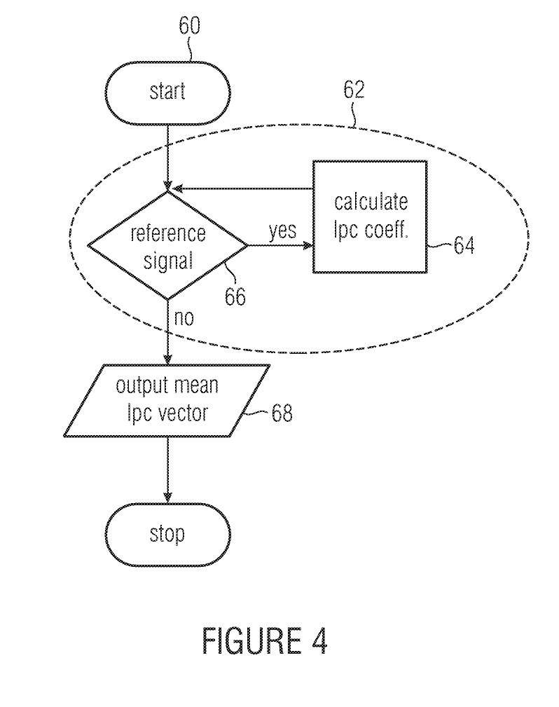

On the basis of FIG. 4, it is shown how reference fingerprint coefficients according to the invention can be determined, on the basis of the example of LPC coding.

The problem to be solved mathematically here is equivalent to the procedure described on the basis of FIG. 1 when detecting the relevant signal areas. Here, a number of reference signals is made available to the algorithm, i.e. such signals identified manually as signals preceding an apnea.

Following a provision step 60, a computation loop 62 begins, in which a set of reference fingerprint coefficients are determined for each reference signal in a computation step 64. If it is determined, during a checking step 66, that no additional reference signals are available anymore, the computation loop 62 is left, and averaged fingerprint coefficients are output as reference fingerprint coefficients, which are calculated in an output step 68, whereupon the execution of the program or method can be terminated.

Although the inventive concept has substantially been described on the basis of LPC coding in the previous embodiments, it is also possible to perform it with any other method of digital speech processing, such as the Hidden-Markov models already described. Here, it is particularly advantageous to use speech-modeling algorithms capable of generating a feature vector of small dimension, in order to implement the method according to the invention in real time and with little computational effort. Here, the speech processing algorithms are especially advantageous particularly because they particularly increase the recognition power due to their following the human vocal organ.

In further embodiments of the present invention, the inventive concept may be supplemented by other criteria increasing the reliability of the recognition. Motivated by the typical waveform of an apnea exemplarily described on the basis of FIG. 5A, an additional criterion may for example consist in the fact that, after a possible beginning of an apnea recognized by means of the fingerprint coefficients, at least a time interval greater than the normal interval of two snoring noises observed until then has to have elapsed without noise before the occurrence of an apnea finally is concluded.

Since the snoring noise accompanies the breathing, no disadvantage regarding the health of the patient is to be expected thereby. It is the advantage, however, that on the one hand there is some additional time margin for performing the signal evaluation, and on the other hand an additional safety criterion is introduced, so that the number of events erroneously classified as the beginning of the apnea can be lowered significantly.

Although the inventive concept does not necessitate that the sample values used for the evaluation are generated in real time, i.e. that a microphone with digitization is connected immediately to the analyzer, this may make sense if the occurrence of an apnea not only is to be detected, but also prevented. Such an apparatus is shown exemplarily on the basis of FIG. 1. Here, the transmission path from the microphone to the sampling means, or from the sampling means to the analyzer can be implemented arbitrarily. In particular, this may be implemented in wireless fashion via common technologies such as WLAN or Bluetooth.

Although the previous figures suggest that the window width used for the analysis of the sample values is default, alternative embodiments in which the window width also is adapted adaptively to the individual patient or self adapts due to the recorded signals are possible.

Depending on the conditions, the inventive method of detecting the beginning of an apnea may be implemented in hardware or in software. The implementation may be on a digital storage medium, particularly a floppy disc or CD with electronically readable control signals capable of cooperating with a programmable computer system so that the inventive method of detecting the beginning of an apnea is executed. In general, the invention thus also consists in a computer program product with program code stored on a machine-readable carrier for performing the inventive method, when the computer program product is executed on a computer. In other words, the invention may thus be realized as a computer program with a program code for performing the method, when the computer program is executed on a computer.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1-18. (canceled)

19. An apparatus for detecting the beginning of an apnea, using a series of sample values determined at predetermined time instants, which describe a breathing noise of a patient, comprising:
an analyzer for analyzing the series of sample values in block-wise manner to determine, for a number of sample values within a block corresponding to a time interval of the breathing noise, a fingerprint with a predetermined number of LPC coefficients describing a waveform of the sample values within the block, with the predetermined number of LPC coefficients being smaller than the number of sample values within the block;
an evaluator formed to recognize the beginning of an apnea by comparison of a vector of the LPC coefficients with a vector of predetermined reference LPC coefficients characteristic of a waveform at the beginning of an apnea, wherein the beginning of an apnea is recognized if the vector of LPC coefficients lies within a tolerance range around the vector of reference LPC coefficients; and
an alarm for performing an alarm action when the evaluator has recognized the beginning of an apnea.

20. The apparatus according to claim 19, wherein the alarm action comprises stimulation of a patient to end the apnea.

21. The apparatus according to claim 19, wherein the analyzer is formed to determine the predetermined number of fingerprint coefficients such that a difference between a linear combination, associated with the sample value, of a number of previous sample values corresponding to the predetermined number of fingerprint coefficients with the fingerprint coefficients as coefficients and the sample value is smaller than a predetermined tolerance value.

22. The apparatus according to claim 19, wherein the analyzer is formed to determine the predetermined number of fingerprint coefficients such that the mean difference of all sample values and the linear combinations associated therewith is minimum for the number of sample values.

23. The apparatus according to claim 19, wherein the tolerance range is a range in which the Euclidian distance of the vector of the LPC coefficients and the vector of the reference LPC coefficients is below a predetermined tolerance value.

24. The apparatus according to claim 19, wherein the analyzer is formed such that the time interval ranges from 100 to 500 ms.

25. The apparatus according to claim 19, wherein the analyzer further is formed to analyze a number of second sample values corresponding to a second time interval, with the time interval and the second time interval overlapping temporally.

26. The apparatus according to claim 19, wherein the analyzer is formed to provide the number of sample values within the time interval with a weight determined individually for each sample value.

27. The apparatus according to claim 19, wherein the analyzer comprises a wireless data interface for receiving the series of sample values.

28. The apparatus according to claim 19, further comprising:
a microphone for recording the breathing noise; and
a quantizer for generating the series of sample values based on the recorded breathing noise.

29. The apparatus according to claim 28, wherein the microphone is a larynx microphone.

30. The apparatus according to claim 28, wherein the quantizer comprises a wireless data interface for transmitting the sample values.

31. The apparatus according to claim 28, wherein the quantizer is formed to quantize the breathing noise at less than 13-bit resolution.

32. A method of detecting the beginning of an apnea, using a series of sample values determined at predetermined time instants, which describe a breathing noise of a patient, comprising:
analyzing the series of sample values in block-wise manner to determine, for a number of sample values within a block corresponding to a time interval of the breathing noise, a fingerprint with a predetermined number of LPC coefficients describing a waveform of the sample values within the block, with the predetermined number of LPC coefficients being smaller than the number of sample values within the block;
comparing a vector of the number of LPC coefficients with a vector of predetermined reference LPC coefficients characteristic for a waveform at the beginning of an apnea, in order to recognize the beginning of the apnea, wherein the beginning of an apnea is recognized if the vector of LPC coefficients lies within a tolerance range around the vector of reference LPC coefficients; and
performing an alarm action at the beginning of an apnea.

33. The method according to claim 32, wherein the alarm action comprises stimulating a patient to end the apnea.

34. A computer readable medium storing a program with a program code for performing, when the program is executed on a computer, a method of detecting the beginning of an apnea, using a series of sample values determined at predetermined time instants, which describe a breathing noise of a patient, the method comprising:
analyzing the series of sample values in block-wise manner to determine, for a number of sample values within a block corresponding to a time interval of the breathing noise, a fingerprint with a predetermined number of LPC coefficients describing a waveform of the sample values within the block, with the predetermined number of LPC coefficients being smaller than the number of sample values within the block;
comparing a vector of the number of LPC coefficients with a vector of predetermined reference LPC coefficients characteristic for a waveform at the beginning of an apnea, in order to recognize the beginning of the apnea, wherein the beginning of an apnea is recognized if the vector of LPC coefficients lies within a tolerance range around the vector of reference LPC coefficients; and
performing an alarm action at the beginning of an apnea.
